(19) United States
(12) Patent Application Publication
(10) Pub. No.: US 2015/0157268 A1
(43) Pub. Date: Jun. 11, 2015

(54) ORGAN WALL RETENTION MECHANISM FOR IMPLANTS

(71) Applicant: VECTORIOUS MEDICAL TECHNOLOGIES LTD., Tel Aviv (IL)

(72) Inventors: Ronny Winshtein, Ramat-Hasharon (IL); Oren Goldshtein, Nahariya (IL); Eyal Orion, Ramat-Efal (IL)

(73) Assignee: VECTORIOUS MEDICAL TECHNOLOGIES LTD, Tel Aviv (IL)

(21) Appl. No.: 14/411,521

(22) PCT Filed: Jul. 1, 2013
(86) PCT No.: PCT/IB2013/001401

§ 371 Date: Dec. 29, 2014

Related U.S. Application Data
(60) Provisional application No. 61/667,990, filed on Jul. 4, 2012.

(51) Int. Cl. A61B 5/00; A61B 5/02; A61B 17/34

(52) U.S. Cl. CPC A61B 5/6882; A61B 5/686; A61B 17/3468; A61B 5/02

(57) ABSTRACT

The application provides systems, devices and methods for anchoring implants within the human body, and more specifically to retention means for attaching sensory and other implants in a heart chamber. Displosed is a sensory implant (1000) comprising: (1) an elongated body (1100) enclosing a lumen and a sensory element (1200) disposed therein; (2) a proximal retention member (1400) coupled to said elongated body and comprising a plurality of projections (1420), each of said projections ending with a projection free end; and (3) a distal retention member (1300) positioned distally to said proximal retention member and coupled to said elongated body, said distal retention member comprising a plurality of legs (1320) originating at a distal end of a base portion, each of said legs ending with a leg free end, said distal retention member is self-expandable from a fully closed formation, in which said plurality of legs are substantially straighten and extend axially to said base portion and distally, to a predetermined non-stressed shape, in which said plurality of legs extend laterally to said base portion and proximally, wherein said distal retention member is configured for tissue ingrowth over said plurality of legs. The implant can be a pressure sensing implant and comprise scraping members to move away tissue.

### TECHNICAL FIELD

The present system, device and method relate to implants connectable to target organs in the human body, and more specifically to retention means for attaching sensory or other implants in a heart chamber and to methods of deployment thereof.

### BACKGROUND

Permanent sensory implants are needed for a variety of illnesses or for preventing heath deteriorations by providing prolonged continuous monitoring. Sensory implants allow real-time patient-specific information for patients with special needs and specific behavior of monitored organs. One such case is pressure monitoring in Congestive Heart Failure (CHF) patients, where efforts are made to develop small sensory implants for monitoring pressure changes in left atrium or in other anatomical locations, in order to provide early and accurate detection of a potential heart function decline.

U.S. Pat. No. 7,899,550 presents trans-septal fixation apparatus and method, describing a “lead implanted across a heart wall such as an atrial septum includes structure for fixation to the wall. In some embodiments the distal end of the lead includes a sensor for measuring quantities such as pressure at a distal side of the wall. The fixation structures may be positioned on opposite sides of the wall after implant. The fixation structures may be aligned with the lead during delivery of the lead to the implant site and expanded from the lead at the implant site.”

U.S. Pat. No. 7,317,951 describes an “anchor and procedure for placing a medical implant, such as for monitoring physiological parameters. The anchor includes a central body in which a medical implant can be received. Arms and members extend radially from first and second ends, respectively, of the central body. Each member defines a leg extending toward distal portions of the arms to provide a clamping action. The anchor and its implant are placed by coupling first and second guidewires to first and second portions of the anchor, placing an end of a delivery catheter in a wall where implantation is desired, inserting the anchor in the catheter with the guidewires to locate the anchor within the wall, deploying the arms of the anchor at one side of the wall followed by deployment of the members at the opposite side of the wall, and thereafter decoupling the guidewires from the anchor.”

It is important to decrease diameter of the sensory implant in order to minimize traumatic effects during implant deployment and improve ease of delivery. Small diameter implants, in the order of 1.5 mm or less, may be introduced in dedicated catheters or delivery needle units which can inject them in-place, for example in open heart or minimally invasive procedures. Such micro sized implants also lessen potential effects to the host organ, for example sensitive or small dimensioned organ walls. In cases that the host organ wall is muscular or otherwise subject to continuous motility, as in a muscular atrial wall, larger sized implants permanently implanted therein or therethrough may affect some functionality and/or integrity of the wall.

In the effort to design and produce a micro sized sensory implant, thought must be made to retention means that connects the implant to a specific point or portion of the organ wall. Such retention means should shift from an extremely low-profile delivery formation to a deployed formation in which they retain the implant in-place and should be able to withstand substantial forces aiming to re-collapse or otherwise deform it.

### SUMMARY OF THE INVENTION

The current invention seeks to provide a system, device and method for implanting an implant in a body organ, using retention means.

In an aspect of some embodiments according to the present disclosure, there is provided a miniature sensory implant having a retention mechanism for anchoring to an organ wall, such as the left atrial wall or the interatrial septum in a human heart. Particular advantages of the present disclosure are included when the retention mechanism is considered (1) for anchoring into a (optionally pulsating) wall portion for a permanent placement and/or long-term (e.g., at least 3 years) effective function, and/or (2) to be delivered via a very small lumen (e.g. equal or less than approximately 2 mm, such as equal or less than 1.5 mm) and/or (3) to allow reversibility (e.g., re-collapsing) during deployment stages, for example in cases where re-positioning is needed.

In some embodiments, optionally considering implant delivery via a small lumen, the retention mechanism includes elastic retention member(s) extendable from a straight form to a laterally extended form; each retention member may include a plurality of projections or legs.

In some embodiments, optionally considering prolonged anchoring to a wall, especially if to a continuously changing (e.g., pulsating) wall or environment, the retention members are designed withstand many/infinite cyclic stresses (e.g., for bending) under normal or expected strains in order to avoid plastic deformation and/or fatigue.

In some embodiments, optionally considering prolonged storage and/or delivery, in which the retention members are fully stressed and/or strained to compress and align, the retention members are designed not to cross maximally permissible strain in order to avoid plastic deformation and/or fatigue.

In some embodiments, retention member design and shape allows elastic re-collapsing, at least during deployment stages, when pulled back into the small lumen of the delivery device.

All these and other factors, when combined, affect certain design factors such as material choice, manufacturing (e.g., machining) consideration, number of formed parts, number of retention members and number of legs/projections in each member, fully-stressed and non-stressed shapes of the retention members and legs/projections, and others.

In some exemplary embodiments, a retention member is created by making a number of evenly spaced slits to a tubular member to form a chosen number of even sized legs/projections, each having a cross-sectional radius of curvature equal to the tubular member's radius. In some such exemplary embodiments, a retention member designed for at least one particular advantage as stated above will include at least one of a minimal number of legs/projections and a minimal length of each leg/projection. In an example where the tubular member is made from a Ni—Ti alloy and having an external diameter smaller than 2 mm, or optionally smaller than 1.5 mm, and having a thickness less than 0.2 mm, or optionally less than 0.1 mm, then the retention member can be formed to include more than four evenly spaced legs/projections, or optionally at least 7 legs/projections, and/or include legs/projections having a length above 4 mm, optionally at least 5.5 mm.

There is thus provided in accordance with the current system, device and method of the present invention a sensory implant comprising an elongated body enclosing a lumen and optionally a sensory element disposed therein, a proximal retention member and a distal retention member positioned distally to the proximal retention member, optionally coupled to elongated body, separately or as a single part. In some embodiments, the proximal retention member includes a plurality of projections, each projection ending with a projection free end. In some embodiments, the distal retention member comprising a plurality of legs originating at a distal end of a base portion, each of the legs ending with a leg free end. In some embodiments, the distal retention member is self-expandable from a fully closed formation (e.g., collapsed configuration), in which the plurality of legs are substantially straighten and extend axially to the base portion and distally, to a predetermined non-stressed shape (e.g., expanded configuration), in which the plurality of legs extend laterally to the base portion and proximally.

In some embodiments, the distal retention member and/or the proximal retention member is formed from metal and free from ingrowth matrix. In some embodiments, the distal retention member and/or the proximal retention member is configured for tissue ingrowth over the plurality of legs and/or projections. Optionally, the base portion includes an outer diameter smaller than 2 mm, optionally smaller than 1.5 mm.

In some embodiments, each the leg free end at the first non-stressed shape is horizontally distant by at least 1 mm from the distal end of the base portion. Optionally, additionally or alternatively, each the leg free end at the first non-stressed shape is vertically distant by at least 3 mm from outer boundaries of the base portion. In some embodiments, the plurality of legs comprising at least 4 legs, optionally at least 7 legs. In some embodiments, the plurality of projections includes at least 4 projections, optionally at least 7 projections.

In some embodiments, the each leg at the first non-stressed shape includes a first curve, the first curve defines a distally projecting medial angle and a proximally projecting lateral angle. Optionally, each leg at the first non-stressed shape includes a second curve lateral to the first curve and curved in opposite direction to the first curve. In some embodiments, the first curve includes a first radius of curvature and the second curve includes a second radius of curvature being substantially greater than the first radius of curvature.

In an aspect of some embodiments of the present disclosure, there is provided a pressure sensing implant with retention members. In some embodiments, the implant includes an elongate body having a proximal end, a distal end, and a lumen therethrough and a pressure sensory element disposed therein. In some embodiments, the implant includes a flexible proximal retention member coupled to the elongate body comprising a plurality of projections, each projection having a free end. In some embodiments, the implant includes a flexible distal retention member coupled to the elongate body comprising a plurality of legs, each leg having a free end.

In some such embodiments, the flexible proximal and distal retention members are self-expandable from a collapsed configuration in which the plurality of projections and legs are substantially straight and the plurality of projections extend proximally of a base portion and the plurality of legs extend distally of the base portion to an expanded configuration in which the plurality of projections extend laterally to the base portion and distally and the plurality of legs extend laterally to the base portion and proximally so as to form symmetrical proximal and distal retention members.

In some embodiments, the flexible proximal and distal retention members are formed from a single retention assembly, wherein the base portion is coupled to the elongate body. In some embodiments, the plurality of projections originates at a proximal end of the base portion and the plurality of legs originates at a distal end of the base portion. Optionally, the proximal retention member comprises at least 7 projections and/or the distal retention member comprises at least 7 legs.

In some embodiments, each leg in the expanded configuration comprises a first curve defining a distally projecting medial angle and a proximally projecting lateral angle and a second curve lateral to the first curve and curved in opposite direction to the first curve. Optionally, the second curve deforms more than the first curve. Optionally, additionally or alternatively, each projection in the expanded configuration comprises a third curve defining a proximally projecting medial angle and a distally projecting lateral angle and a fourth curve lateral to the third curve and curved in opposite direction to the third curve. Optionally, the fourth curve deforms more than the third curve.

In some embodiments, the plurality of projections and legs form predetermined spider leg shapes.

In some embodiments, the plurality of projections and legs in the expanded configuration are identical in number and dimension.

In some embodiments, the pressure sensory element extends along a side and in proximity to a distal end of the elongate body. Optionally, the pressure sensory element is positioned distally of the flexible distal retention member in the expanded configuration. In some embodiments, the pressure sensory element comprises a pressure transducer having a membrane sensitive to pressure changes.

In an aspect of some embodiments of the present disclosure, there is provided a pressure sensing implant with retention and scraping members. In some embodiments, the implant includes an elongate body having a proximal end, a distal end, and a lumen therethrough and a pressure sensory element disposed therein. In some embodiments, the implant includes a proximal retention member coupled to the elongate body comprising a plurality of projections, each projection having a free end. In some embodiments, the implant includes a distal retention member coupled to the elongate body comprising a plurality of legs, each leg having a free end.

In some such embodiments, the flexible proximal and distal retention members are self-expandable from a collapsed configuration in which the plurality of projections and legs are substantially straight and extend axially to a base portion and distally to an expanded configuration in which the plurality of projections extend laterally to the base portion and distally and the plurality of legs extend laterally to the base portion and proximally, wherein the proximal projections are configured to scrape or move away an intermediate structure from a target site.

In some embodiments, the free end of each proximal projection is configured to scrape or move away the intermediate structure. In an aspect of some embodiments of the present disclosure, there is provided a method comprising at least one of the following steps (not necessarily in same order):
locating a wall portion of an heart atrium;
delivering an implant to a target site on an external surface of the wall portion, the implant provided in a tubular member, the tubular member comprising a lumen opened at a distal end thereof and sized for maintaining the distal retention member at the fully closed formation, the implant is releasably connected to a pusher;
penetrating with the tubular member through the target site into the heart atrium;
protruding the implant partially outside the lumen using the pusher and/or the tubular member to release the distal retention member from the fully closed formation; and
verifying deployment by applying an axial pulling force smaller than substantially 250 grams to the pusher.

In some embodiments, the method includes a step of transferring the implant fully outside the lumen using the pusher to release the proximal retention member from the fully collapsed formation.

In some embodiments, the method includes a step of retracting the implant from the heart atrium into the lumen by applying a pulling force greater than 100 grams, or optionally greater than 250 grams, to the pusher.

In some embodiments, the wall portion is part of a left atrial wall, or optionally part of an interatrial septum and the heart atrium is a left atrium.

In some embodiments, delivering the implant to the target site includes perforating a right atrial wall and passing the tubular member through the perforation into a right atrium.

In some embodiments, the method includes at least one of the following steps:
withdrawing the tubular member back through the perforation; and
sealing the perforation.

In some such embodiments, sealing of the perforation includes deploying a closure device in or adjacent the perforation. Optionally, the closure device is or includes a second implant similar or identical to the first implant.

In an aspect of some embodiments there is provided a method for deploying sensory implant (e.g., a pressure sensing implant) with proximal and distal retention members to a target site separated by an intermediate structure, the method comprises at least one of the following steps (not necessarily in same order):
penetrating through an intermediate structure and target site into a heart atrium with a delivery device which receives and maintains a pressure sensing implant with proximal and distal retention members in a collapsed configuration;
deploying the distal retention members from the delivery device so that they self-expand from the collapsed configuration in which the distal retention members are substantially straight and extend distally of a base portion to an expanded configuration in which the distal retention members extend laterally to the base portion and proximally;
engaging the distal retention members to a first surface of the target site;
deploying the proximal retention members from the delivery device so that they self-expand from the collapsed configuration in which the proximal retention members are substantially straight and extend distally to the base portion to the expanded configuration in which the proximal retention members extend laterally to the base potion and distally;
scraping or moving a portion of the intermediate structure away from a second surface of the target site with the proximal retention members; and
engaging the proximal retention members to a second surface of the target site.

In some embodiments, the target site comprises a left atrial wall. Optionally, the first surface comprises an inner surface of the atrial wall and the second surface comprises an outer surface of the atrial wall. Optionally, the intermediate structure comprises fat or connective tissue and/or an organ and/or a left atrial appendage.

In an aspect of some embodiments there is provided a method for redeploying a pressure sensing implant with proximal and distal retention members, the method comprises at least one of the following steps (not necessarily in same order):
penetrating through a first puncture site into a heart atrium with a delivery device which receives and maintains a pressure sensing implant with proximal and distal retention members in a collapsed configuration;
deploying the distal retention members from the delivery device so that they self-expand from the collapsed configuration in which the distal retention members are substantially straight and extend distally of a base portion to an expanded configuration in which the distal retention members extend laterally to the base portion and proximally;
re-collapsing the distal retention members by pushing the delivery device distally over the distal retention members;
pulling the delivery device proximally out of the first puncture site;
penetrating through a second puncture site with the delivery device; and
redeploying the distal retention members from the delivery device.

In some embodiments, the method urther comprising deploying the proximal retention members prior to or after re-collapsing step.

In some embodiments, the first or second puncture size is 2 mm or less in diameter. In some embodiments, the implant size is 1.5 mm or less in diameter.

In some embodiments, the first puncture site naturally seals. In some embodiments, the first or second puncture site comprises a left atrial wall or interatrial septum wall.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-1F schematically illustrate an exemplary implant with retention means shown in different views and formations, in accordance with exemplary embodiments of the present invention;

FIG. 2A-2B schematically illustrate another exemplary implant with another retention means shown in a predetermined non-stressed shape, in accordance with exemplary embodiments of the present invention;

FIG. 3 schematically illustrates an exemplary retention device for housing implants, in accordance with exemplary embodiments of the present invention;

FIGS. 4A-4B schematically illustrate an exemplary implant implanted through organ walls differentiated by wall thickness, in accordance with exemplary embodiments of the present invention;

FIG. 5 schematically illustrates an exemplary implant with a distal retention member deformed to a substantially transverse planar shape under proximally pulling force, in accordance with exemplary embodiments of the present invention;

FIG. 6 schematically illustrates an exemplary implant implanted through an organ wall shown in a shifted position under a lateral force, in accordance with exemplary embodiments of the present invention;

FIGS. 7A-7B schematically illustrate an exemplary implant with retention members at different deployment stages via a needle type delivery device, in accordance with exemplary embodiments of the present invention;

FIG. 8 schematically illustrates an exemplary model for delivering an implant directly to a left atrial wall portion, in accordance with exemplary embodiments of the present invention;

FIG. 9 schematically illustrates an exemplary model for delivering an implant with a catheter to an interatrial septum portion, in accordance with exemplary embodiments of the present invention; and

FIGS. 10A-10B schematically illustrate exemplary steps of an exemplary model for delivering an implant to an interatrial septum portion through the right atrial wall, in accordance with exemplary embodiments of the present invention;

FIGS. 11A-11G schematically illustrate deliovery and deployment of the cantilevered, symmetrical retention device of FIGS. 2 and 3, in accordance with exemplary embodiments of the present invention;

FIGS. 12A-12F schematically illustrate deploying a pressure sensing implant with proximal and distal retention members, such as the implant of FIG. 1A, to a target site separated by an intermediate structure in accordance with an embodiment of the present invention; and

FIGS. 13A-13D schematically illustrate redeployment of an implant/retention device in accordance with an embodiment of the present invention.

### DESCRIPTION OF EMBODIMENTS OF THE INVENTION

The following preferred embodiments may be described in the context of exemplary cardiovascular related sensory implants implantations for ease of description and understanding. However, the invention is not limited to the specifically described devices and methods, and may be adapted to various clinical applications without departing from the overall scope of the invention, for example implantations of sensory implants in other regions or internal organs of the body and/or implantations of other non-sensory implants (e.g., in a cardiovascular organ or in any other internal body organ).

It is to be understood that the terminology used herein is used for the purpose of describing particular embodiments only, and is not intended to limit the scope of the invention. Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

The term “patient” as used herein refers to a mammalian individual afflicted with or prone to a condition, disease or disorder as specified herein, and includes both humans and animals.

The term “sensory implant” as used herein refers to an artifact which includes a sensor or a sensing mechanism designed to receive a signal or stimulus and responds to it in a distinctive manner. The signal or stimulus can be a change in condition and/or a performance of an internal body organ (for example, a change in pressure, temperature, PH or others). The sensory implant may also include other means, optionally provided in an electrical circuit, designed to generate readable or measurable information corresponding to received signal or stimulus and/or designed to transmit a digital signal correlative to the change in condition and/or performance. The sensory implants of this invention may be considered “micro-” or “micro sized” in the sense they are limited in size, and more particularly characterized in a maximal diameter of 2 mm or less, and in some instances of 1.5 mm or less. The sensory implants may be of any length, usually depending optionally in the range of 1 to 30 mm, optionally in the range of 10 to 20 mm.

The term “Organ” or “body organ” as used herein refer to a collection of tissues joined in structural unit to serve a common function. The term “internal body organ” as used herein refers to organs, usually chambers or conduits in a patient's body enclosed with a wall that are commonly positioned distally to a skin tissue and/or muscle tissue and/or bone tissue. Internal body organs may include, but are not limited to, the heart and chambers thereof, veins, arteries, brain, lung, kidney, muscles, ureter, bladder, urethra, mouth, esophagus, stomach, small and large intestines.

The term “wall” as used herein as in a “wall target” or “internal body organ wall” refers to the barrier of the internal body organ, either completely or only partially covering it, having a thickness, and comprising a soft tissue (connective and/or muscular). For example, a wall of a heart chamber will commonly include several layers of soft tissues, including: external fibrous layer, parietal pericardium, visceral pericardium, myocardium and endocardium. An “external surface” of a body organ refers to the overlying surface of the wall at the exterior of the internal body organ. A “wall target” as used herein refers to an area or a point adjacent, on or in the wall or the external surface, to which an implant is carried prior to or as part of deployment and implantation in the wall at the site of implantation. The term “site of implantation” as used herein refers to the physical location where an implant, such as a sensory implant, is inserted into a wall of a body organ and fixedly anchored thereto.

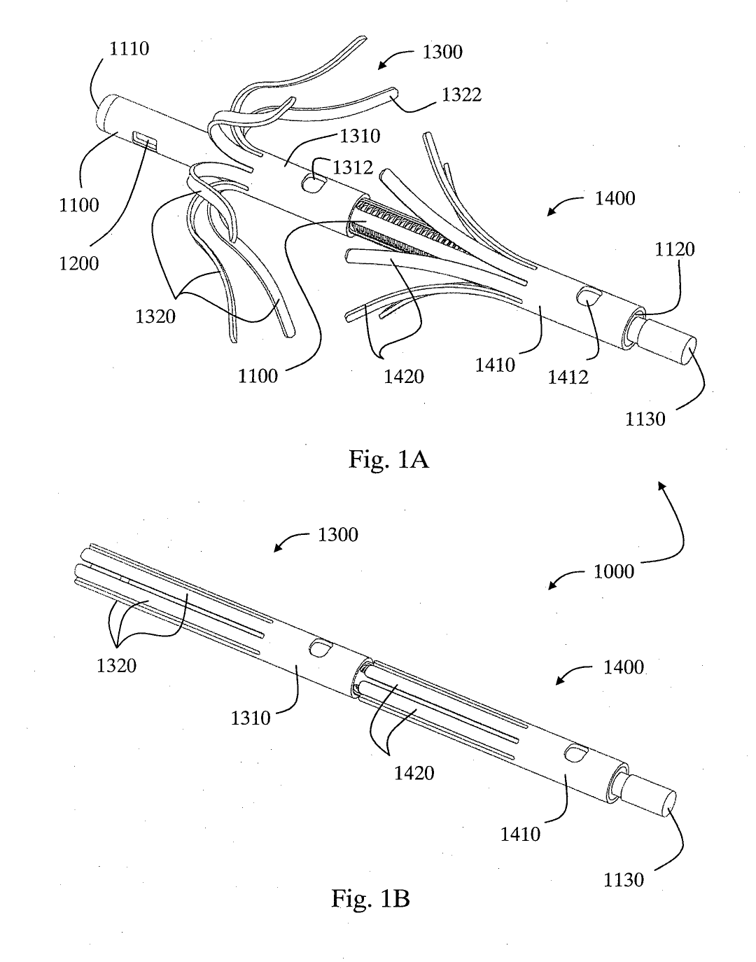

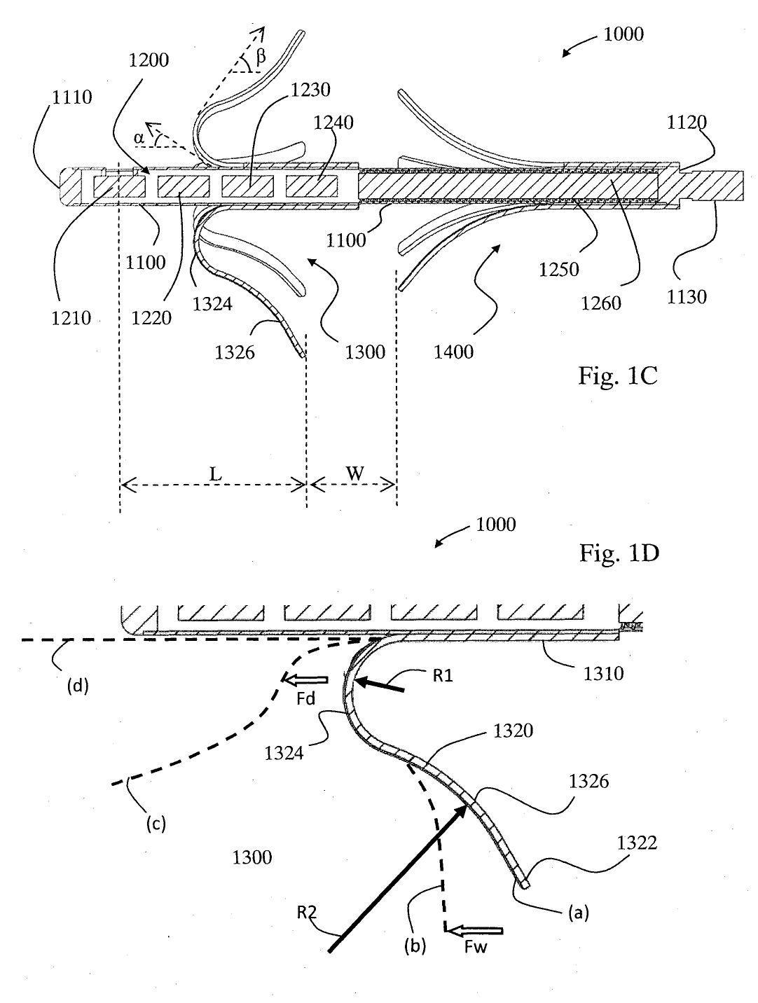

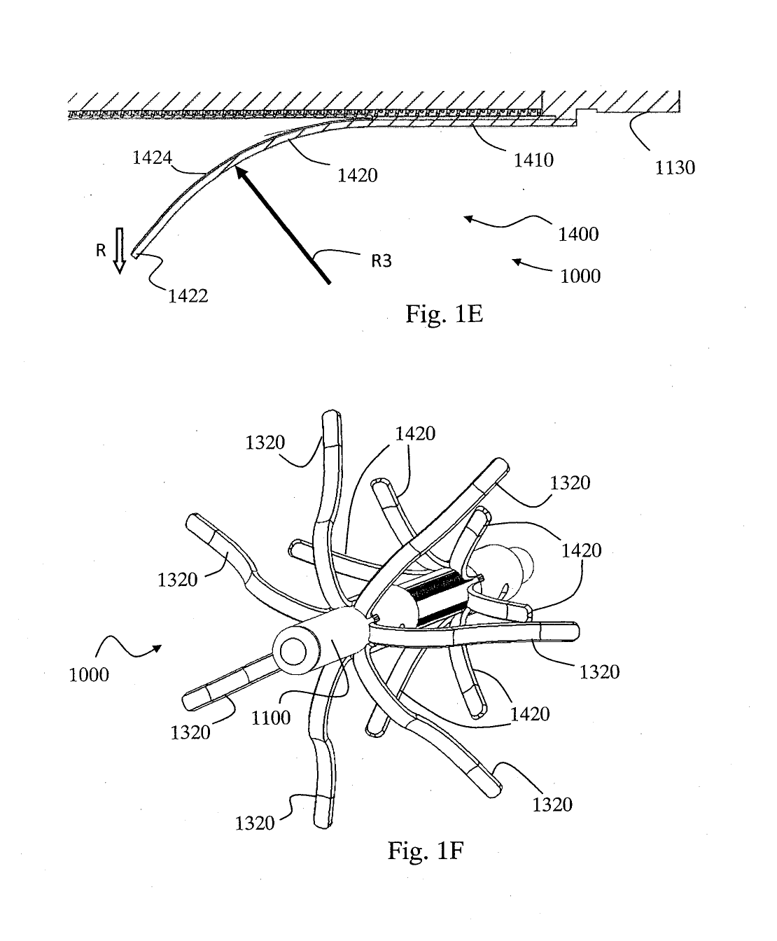

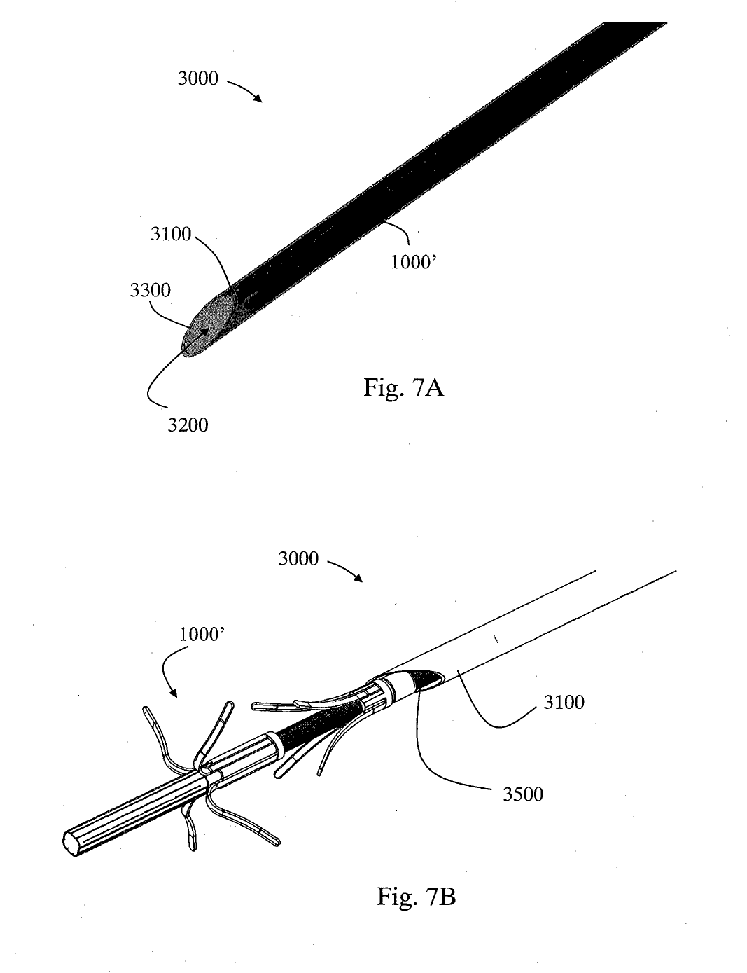

Reference is now made to FIGS. 1A-1F which schematically illustrate an exemplary implant 1000 with retention members 1300 and 1400 shown in different views and formations. FIG. 1A shows implant 1000 in a predetermined non-stressed shape in an expanded configuration, FIG. 1B shows implant 1000 in a fully closed formation, FIG. 1C shows a cross sectional view of implant 1000, FIG. 1D shows a partial cross sectional view of distal retention member 1300 and FIG. 1E shows a partial cross sectional view of proximal retention member 1400. FIG. 1F illustrates another isometric view of implant 1000 in the expanded configuration. As shown, implant 1000 includes an elongated body 1100 having a distal end 1110 and a proximal end 1120. Elongated body 1100 also includes an optional connecting member 1130 adapted for releasably/detachably connecting (e.g., by threading) with delivery means, such as a pusher (e.g., pusher 3500 shown in FIG. 7B provided in a sheath 3100 as part of a delivery device 3000). Elongated body 1100 may be at least 5 mm in length, optionally at least 10 mm, optionally at least 15 mm, optionally at least 20 mm, or higher, or lower, or intermediate to these values. In a fully closed formation, retention members 1300 and 1400 are aligned, both is same direction (e.g., distal direction, as shown in FIG. 1B), and define an outer diameter that is smaller than 2 mm, optionally smaller than 1.5 mm, optionally 0.8-1.2 mm, optionally about 1.1 mm, or higher, or lower, or any intermediate value.

Implant 1000 may be a sensory implant and as such be optionally configured for prolonged and continuous and/or sequential measurements of at least one parameter of interest in a body organ, such as a heart, and optionally more specifically a left atrium in the heart. In some embodiments, implant 1000 is configured for pressure measurements, for example in CHF patients or other patients in need for personalized monitoring of pressure changes in the heart or elsewhere. Elongated body 1100 houses a measurement unit 1200, including a sensory element 1210 provided in proximity to a distal end 1110 of elongated body 1100. Sensory element 1210 may be sensitive to pressure changes and in some embodiments may include or be part (e.g., a membrane) in a pressure sensing micro electro-mechanical system (MEMS). Optionally and alternatively, implant 1000 may be another type of device or apparatus, for example, an anchor (e.g., for other devices, tethers, sutures or others), an electrode, a drug delivery device, or another.

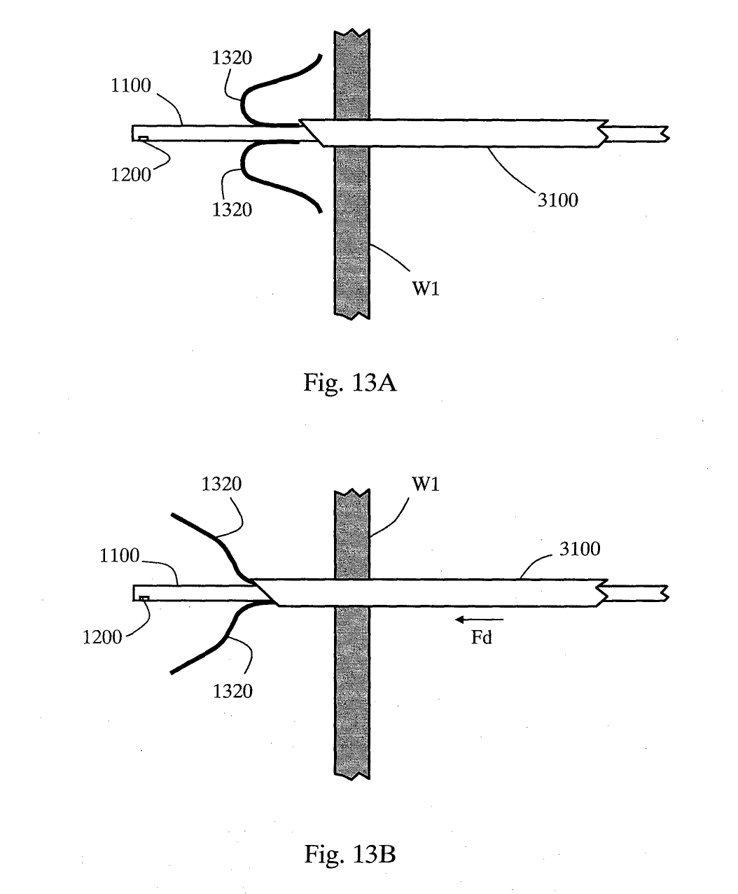

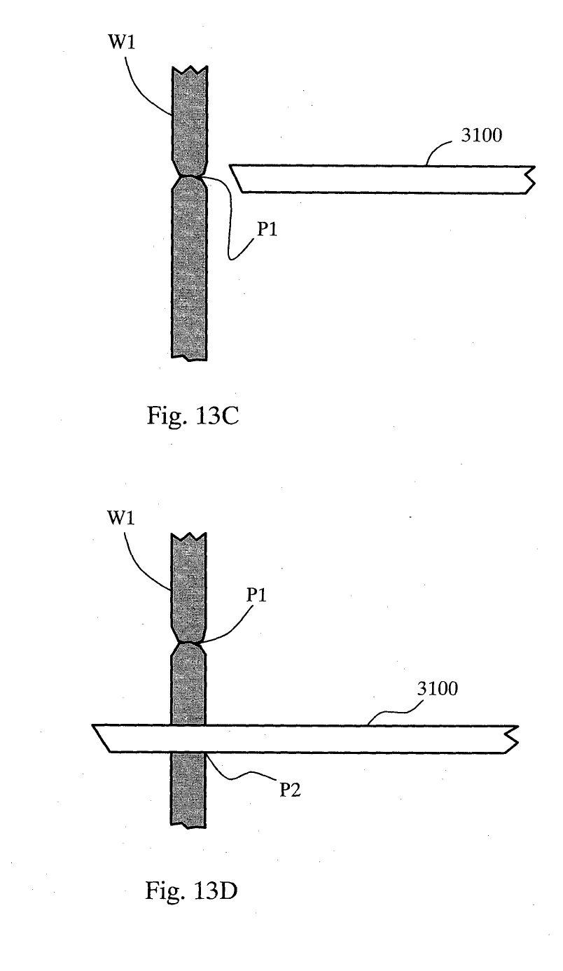

Distal retention member 1300 includes a plurality of legs 1320 which originate at a distal end of a distal base portion 1310. In some such embodiments, each leg 1320 ends with a leg free end 1322. Distal retention member is self-expandable from a fully closed formation (as shown in FIG. 1B), in which legs 1320 are substantially straighten and extend axially to base portion 1310 and distally, to a predetermined non-stressed shape, in which legs 1320 extend laterally to base portion 1310 and proximally. In some embodiments, implant 1000 is designed for optional withdrawing from an original implantation site in a wall target and relocating to another, for example in order to improve safety of efficacy of the implant by choosing a more appropriate anatomical location, optionally patient-specific, as the final implantation site, optionally after at least one previous attempt. In some such embodiments, legs 1320 are designed and configured for recollapsing into a delivery device (as shown in FIGS. 13A-13D). As shown in FIG. 1A, at the non-stressed shape, legs 1320 in distal retention member 1300 take a form similar to “spider's legs” which provides improved elasticity, especially in longitudinal axis direction, as well as capability to recollapse into a tubular member (such as, for example, sheath 3100) by realigning and straightening distally as shown in FIG. 1B.

In some embodiments (shown in FIGS. 1C-D), each leg 1320, at the non-stressed shape, includes a first curve 1324, which defines a distally projecting medial angle a and a proximally projecting lateral angle β. Since that lateral angle β projects proximally in opposite direction to medial angle α, sheath 3100, if pushed distally against first curve 1324 under sufficient force, such as recollapsing force Fd applied by a tubular member or a sheath (shown in FIG. 1D), will cause legs 1320 to recollapse. In some embodiments, each leg 1320 at its non-stressed shape also includes a second curve 1326 lateral to first curve 1324 and curved in opposite direction to first curve 1324. As shown in FIG. 1D, first curve 1324 may include a first radius of curvature R1 and second curve 1326 includes a second radius of curvature R2 being substantially greater than first radius of curvature R1. As such, each legs 1320 is prone to elastically deform along second curve 1326 more than along first curve 1324, at least when distally projecting forces are applied thereto at its free end 1322, such as force Fw applied by the wall target. Therefore, second curve 1326 is designed such that it contributes most to leg elasticity needed for implant retention in a wall target, for example to a moving (e.g., muscular and/or pulsatory) tissue such as the left atrial wall, while, optionally, first curve 1324 contributes most to leg recollapsing function at need. One of the advantages in the proposed design lies in the axial distance of leg free ends 1322 to their origin at base portion 1310. Each free end 1322 is positioned, at the non-stressed shape, proximally to its origin, allowing it to deform and/or tilt backwards (i.e., distally) while elastically resisting motion during a substantial travel before re-collapsing is unavoidable as deriving from the geometry of the distal retention member 1300.

FIG. 1D summarizes four optional exemplary formations of leg 1320 in view of different situations:

“Formation (a)”—in which leg 1320 is in the predetermined non-stressed formation as previously described. In this scenario, no substantial external forces and internal stresses are present in leg 1320 hence it is positioned, contoured and shaped as set during manufacturing (e.g., heat treating and/or cold work).

“Formation (b)”—in which organ wall presses (either dynamically and/or passively) leg 1320 distally by applying a force Fw to free end 1322 to take approximately the suggested tilt and shape. In some embodiments, when forces Fw applied to leg 1320 being under 200 gr, optionally under 100 gr, optionally under 50 gr, optionally under 20 gr, second curve 1326 will deform substantially more than first curve 1324.

“Formation (c)”—in which a delivery device (as shown in FIG. 13B), optionally a tubular member or a sheath (e.g., sheath 3100), is pushed distally against leg 1320 in the area of first curve 1324 by a force Fd enough to deform entire leg 1320 to approximately as shown.

“Formation (d)”—in which leg 1320 is completely collapsed, straightened and pointed distally. Such a scenario may occur if implant 100 is completely withdrawn into a sheath of a delivery device, optionally by first applying distally oriented force Fd. Optionally, in order to completely collapse leg 1320 as shown, Fd is greater than 50 grams, optionally greater than 100 gr, optionally greater than 300 gr, or higher, or lower, or intermediate.

Similarly to distal retention member 1300, proximal retention member 1400 includes a plurality of projections 1420 projecting from a proximal base portion 1410. In some such embodiments, proximal retention member 1400 is self-expandable from a fully collapsed formation to a second predetermined non-stressed shape (e.g., expanded configuration). In some embodiments, projections 1420, when in the fully collapsed formation/configuration, are substantially straighten and extend axially to the proximal base portion 1410 (optionally also to distal base portion 1310) and distally, and in the second predetermined non-stressed shape, projections 1420 extend laterally to proximal base portion 1410 and distally. In some such embodiments, each projection 1420, when at the second non-stressed shape, is substantially straight or is curved in a single direction only, and, optionally, includes a single curve 1424 having a radius of curvature R3 (as shown in FIG. 1A). This design for distal retention member 1400 is substantially different than the design of distal retention member 1300 as it provides more stiffness and/or elasticity for each projection 1420 in the lateral direction. As shown in FIG. 1E, projection 1420 has lateral elastic resistance R to compressive forces, which may resist collapsing under compressive forces being 5 grams or less, optionally 10 grams or less, optionally 20 grams or less, optionally 40 grams or less, optionally 100 grams or less, or higher, or lower, or any intermediate value. This can be useful for example if implant 1000 is deployed such that proximal retention member 1400 is placed at the outer surface of an organ wall being adjacent to other organs or tissues and/or subject to disturbances by external forces other than those made by or through the wall. Therefore, as oppose to the “spider's legs” type formation of distal retention member 1300, proximal retention member 1400 has a “scraper” type formation, as projections 1420 are designed to move aside and/or to scrap away organs or tissues at its premises. For example, portions of the outer surface of the left atrial wall in a human heart are commonly covered with substantial volume of fat tissue and/or other connective or soft tissue, making it more difficult to fasten to the wall target and to open axisymmetrically. Furthermore, an adjacent left atrial appendage may limit lateral expansion of common self-expandable anchors. Hence the use of the “scraper” design for proximal retention member 1400 allows improved and forceful opening and maintaining of such opening even under substantial stresses.

As previously described, implant 1000 may be a sensory implant having a lumen for containing a sensory element 1210 (e.g., a pressure transducer comprising a membrane sensitive to pressure changes). In some such embodiments, as shown in FIG. 1C, implant 1000 houses an entire measurement unit 1200 comprising, other than sensory element 1210, at least one of a capacitor 1220 (needed, for example, in case that sensory element 1210 is a capacitive based MEMS transducer), at least one electrical component 1230 (for example, any of a telemetry unit, a motherboard, a memory, a battery, an amplifier, an antenna, a sensor, or other), an application-specific integrated circuit (ASIC) 1240, adapted to convert the MEMS capacitance to a frequency-encoded signal, a transmitter and/or antenna 1250 designed to signal data to a remote receiver (not shown) provided outside patient's body, allowing a wireless connection for transmitting sensed data in real-time, and means for collecting remote powering such as a power receiver 1260 configured for receiving powering energy transmitted wirelessly from a remote source.

In some embodiments, sensory element 1210 is provided in proximity to distal end 1110 of elongated body 1100. In some such embodiments, it is found significant to position sensory element 1210 in a body chamber such as an atrium, distally and remotely away from inner surface of the organ wall, so that generation and/or accumulation of tissue or aggregations covering the sensory element will be diminished or prevented, hence its ability to further function continuously for prolonged duration with smaller or minimized effect to sampling accuracy and/or drift. In some embodiments, distal retention member 1300 and/or proximal retention member 1400 are designed such that, upon deployment, sensory element 1210 is distanced from organ wall by at least 1 mm, optionally at least 2 mm, optionally at least 4 mm or higher, or lower, or any intermediate distance. It is further advantageous to position the sensory element 1210 along a side portion and towards the distal end 1110 of the elongated body 1100 to reduce a pulsatile coupling effect due to implantation within highly motile heart wall tissue.

Similarly, it is advantageous to extend signal transmitter 1250 and/or power receiver 1260 along a substantial length of elongated body 1100 and/or locate any of them at least partly outside the organ adjacent proximal end 1120. Therefore, according to some embodiments, sensory element 1210 is positioned distally to the legs, and more particularly at least 3 mm horizontally distant to free ends 1322 of legs 1320 when the are at said first non-stressed shape, optionally at least 5 mm, optionally at least 7 mm, or higher, or lower, or in any intermediate value. In some such embodiments, if legs 1320 at deployment are stretched distally, a minimally allowed distance L of at least 1 mm distally to free ends 1322, optionally at least 3 mm, is optionally applied. Optionally, additionally or alternatively, there is a calculated linkage between the minimally allowed distance L and the final distance or width W between distal legs free ends 1322 and proximal projections free ends 1422. For example, if a wall target has a width W greater than distance between legs free ends 1322 and proximal projections free ends 1422, at non-stressed formations, by 1 mm, for example if width W is approximately 3 mm, then distance L at said width W is optionally greater than 3 mm, for example approximately 4 mm.

As implant 1000 is designed for delivery in micro sized dimensions, preferably 1.5 mm or less in diameter, while allowing retention members 1300 and 1400 to expand and effectively retain it in-place even under substantial disturbances during deployment and/or afterwards, specific materials, design factors and dimensions are preferred. One optional design factor includes the use of metals in super-elastic conditions, such as Ni—Ti based alloys, for the distal and/or proximal retention members. In some embodiments, plurality of legs 1320 is configured for infinite continuous suppression to the fully closed formation with negligible plastic deformation. Ni—Ti alloys based retention members commonly allow maximal permissible strain below 8% without plastic deformation, therefore the retention members should be designed such that when fully deformed from a non-stressed formation (for example, when a self-expandable retention member is in a fully closed/collapsed formation) the maximal strain developed therein will be substantially less than 8%, for example 7% or less, or 6% or less. Furthermore, Ni—Ti alloys based retention members commonly allow a maximal cyclic strain below 0.7% for virtually indefinite cyclic stresses without fatigue, therefore the retention members should be designed such that in maximal encountered cyclic tilt (for example, the tilt shown in “Formation (b)” in FIG. 1D), the maximal cyclic strain developed therein will be substantially less than 0.7%, for example 0.6% or less, or 0.5% or less at maximal cyclic tilts of 20% or less, optionally 10% or less.

Another optional design factor includes the use of a minimal amount of metal pieces for forming as few as possible implant structural parts. In a first exemplary embodiment, implant elongated body 1100, distal retention member 1300 and proximal retention member are formed as a single structural part; in a second exemplary embodiment, elongated body 1100 is made as a first single structural part and distal retention member 1300 and proximal retention member 1400 are made as a second single structural part that is fixated (e.g., glued, welded or soldered) to or over elongated body 1100; and in a third exemplary embodiments, each of these three elements is formed as a single structural member. As shown in FIG. 1A, distal retention member 1300 is made of a single metal piece as a single structural member and is covering a distal portion of elongated body 1100 and fixated thereto via at least one opening 1312 provided in distal base portion 1310; whereas proximal retention member 1400 also is made of a single metal piece as a single structural member and is covering a proximal portion of elongated body 1100 and fixated thereto via at least one opening 1412 provided in proximal base portion 1410.

Yet another design factor includes the use of maximally possible amount of metal for the retention members, or at least the distal retention member. Therefore, minimal number and size of cuts and slits are optionally made, while keeping in mind the other design factors and restraints, as for example, there may be a tradeoff between number of slits (therefore, optionally, number of legs or projections) and lowering estimated maximal and/or cyclic strains to substantially below permissible values. FIG. 1F illustrates an exemplary implant 1000 with at least seven legs 1320 and seven projections 1420 for secure anchoring within a pulsating heart wall portion over long periods of time (e.g., months to years).

Therefore, and according to some exemplary embodiments, any of distal retention member 1300 and proximal retention member 1400 is formed from a single metal piece and/or is formed as a single structural part. In some such embodiments, the single piece metal and/or single structural member is tubular and optionally the forming includes creating longitudinal slits 0.2 mm or less wide, optionally 0.15 mm or less, optionally 0.1 mm or less, or lower, or higher, or in any intermediate value. Optionally, each two adjacent slits define a leg. Optionally, the single piece metal is a Ni—Ti alloy in a super-elastic condition.

In some embodiments, elongated body 1100 or distal base portion 1310 and/or proximal base portion 1410 has an outer diameter smaller than 2 mm, optionally smaller than 1.5 mm, optionally 0.8-1.2 mm, optionally about 1.1 mm, or higher, or lower, or any intermediate value. Optionally, additionally or alternatively, distal retention member 1300 or any of its base portion 1310 and/or legs 1320, and/or proximal retention member 1400 or any of its base portion 1410 and/or legs 1420, has a maximal thickness smaller than 0.2 mm, optionally equal or smaller than about 0.1 mm, or higher, or lower, or any intermediate value. In some embodiments, each leg free end 1322 is horizontally distant by at least 1 mm from a distal end of distal base portion 1310 and/or is vertically distant by at least 3 mm from outer boundaries of base portion 1310, when at its non-stressed shape.

In some embodiments, distal retention member 1300 and/or proximal retention member 1400 are tubular, hence each leg 1320 and/or projection 1420, respectively, has curved cross section with a radius of curvature identical to the tube radius. When legs 1320 and/or projections 1420 are forced to deform from non-stressed formations, developed strain can be proportional to several factors such as radius of curvature of leg/projection cross section, as well as leg/projection width, thickness and length. In some embodiments, at certain cross sectional radius of curvature, it is preferable to use a relatively large number of legs and/or projections having smallest possible width and thickness and largest possible length. In some such embodiments, these factors are limited by the strength and elasticity needed for each leg and/or projection. In some exemplary embodiments, plurality of legs 1320 and/or projections 1420 comprise at least 4 legs, optionally at least 6 legs, for example, 7 identical legs (as shown in FIG. 1). Optionally, each leg 1320 and/or projection 1420 is 0.01-1 mm wide, optionally 0.1-0.5 mm wide, optionally about 0.4 mm wide. In some embodiments, legs 1320 at a fully closed formation are at least 2 mm in length, optionally at least 4 mm in length, optionally at least 6 mm in length, optionally at least 10 mm in length, or higher, or lower, or in any intermediate value.

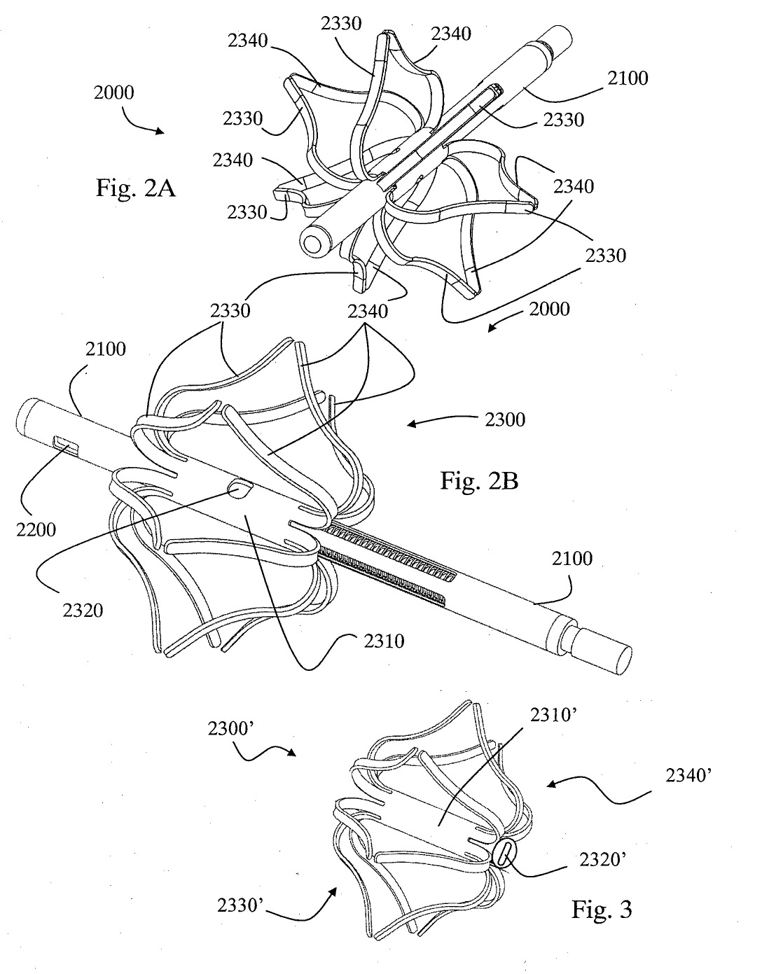

Reference is now made to FIGS. 2A-2B, which schematically Illustrate another exemplary implant 2000 with different retention means shown in a predetermined non-stressed shape. Implant 2000 includes an elongated body 2100, an optional measurement unit 2200 enclosed in elongated body 2100, a single retention member 2300 comprising a base portion 2310 fixated to elongated body via an opening 2320, a plurality of legs 2330 emerging at distal end of base portion 2310 and a plurality of projections 2340 emerging at a proximal end of base portion 2310. In some embodiments, legs 2330 and projections 2340 are identical in number, dimensions and mechanical properties, both formed as “spider's legs”, though positioned in opposite directions in order to provide retention to implant 2000 in a wall target provided there between. In some embodiments, both legs 2330 and projections 2340 self-expand from a fully collapsed formation to a predetermined non-stressed shape. In some such embodiments, when in a fully collapsed formation, projections 2340 are substantially straighten and extend axially to base portion 2310 and proximally, and in the predetermined non-stressed shape projections 2340 extend laterally to base portion 2340 and distally. Optionally, each projection 2340, when at the non-stressed shape, includes a third curve which defines a proximally projecting medial angle and a distally projecting lateral angle. Optionally, each projection 2340 at the non-stressed shape includes a fourth curve lateral to the third curve and curved in opposite direction to the third curve. Optionally, the third curve includes a third radius of curvature and the fourth curve includes a fourth radius of curvature being substantially greater than the third radius of curvature. As described above with reference to FIG. 1D, each projection 2340, like each leg 1320, is prone to elastically deform along the fourth curve more than along the third curve, at least when proximally projecting forces are applied thereto at its free end. As such, each projection 2340 has similar advantages (e.g., elasticity for implant retention in wall target) as those described with reference to leg 1320. FIG. 2A is an isometric view of implant 2000 in the expanded configuration illustrating at least seven legs 2330 and seven projections 2340 for secure anchoring over long durations of time in highly motile heart wall tissue.

FIG. 3 schematically illustrates an exemplary retention device 2300′ for housing implants. Retention device 2300′ includes a base portion 2310′ with a lumen 2320′ provided at least partly along its length and opened in at least a proximal side thereof. A plurality of legs 2330′ emerges from the distal end of base portion 2310′, while a plurality of identical yet opposite projections 2340′ emerges from its proximal side, and self-expands to a non-stressed formation as shown in the figure. In some embodiments, different types of implants can be sized and configured for placement in lumen 2320′, for example a sensory implant, an electrode connected or connectable to electric device such as a pacemaker or a defibrillator, an anchor, a tether, a suture, a drug delivery device, a reservoir, etc.

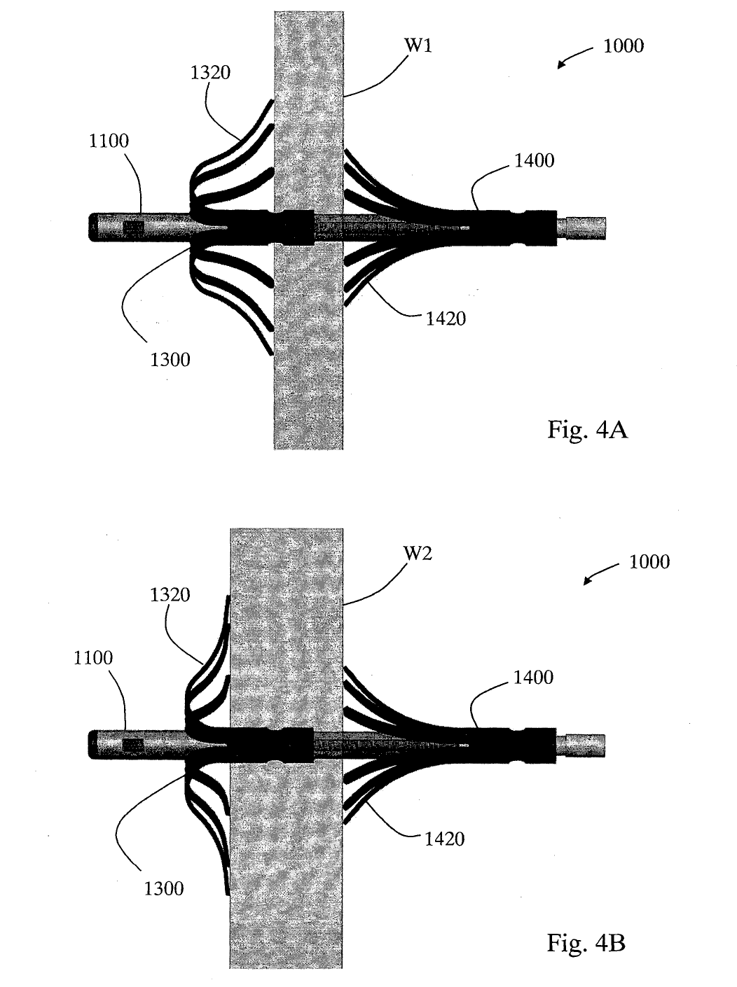

Reference is now made to FIGS. 4A-B which schematically illustrate implant 1000 implanted through organ walls differentiated by wall thickness: in FIG. 4A implant 1000 is implanted through a thinner wall having a thickness W1 and in FIG. 4B implant 1000 is implanted through a thicker wall having a thickness W2. In an exemplary embodiment, thinner wall is an interatrial septum and thickness W1 is commonly between 0.5 mm and 2 mm, optionally about 1 mm, whereas thicker wall is a left atrial wall and thickness W2 is commonly between 2 mm and 5 mm, optionally about 3.5 mm. In some embodiments, legs 1320 free ends are distant by 2 mm or less, or optionally 1 mm or less, from projections 1420 free ends, when in a non-stressed shape. As shown in FIG. 4A, at wall thickness W1 being optionally about 1 mm or about 2 mm, both legs 1320 of distal retention member 1300 and projections 1420 of proximal retention member 1400 are substantially in their non-stressed formation, as W1 is substantially similar to distance between opposite free ends at non-stressed formation. As shown in FIG. 4B, at wall thickness W2 being optionally about 3.5 mm, legs 1320 are further stretched laterally and distally while projections 1420 are substantially in their non-stressed formation, since that W2 is substantially greater than the distance between opposite free ends at their non-stressed formation, and since that legs 1320 are substantially less stiff and bend more easily with respect to projections 1420, at least during relatively small strains as shown in FIG. 4B. In some such embodiments, it may be advantageous to use implants 1000 having distance between opposing distal ends being substantially smaller than actual wall target thickness in a predetermined value, in order to create elastic preloading to legs 1320. Under preloading, legs 1320 will be subject to a greater degree of continuous strain but to a lesser degree of cyclic strains.

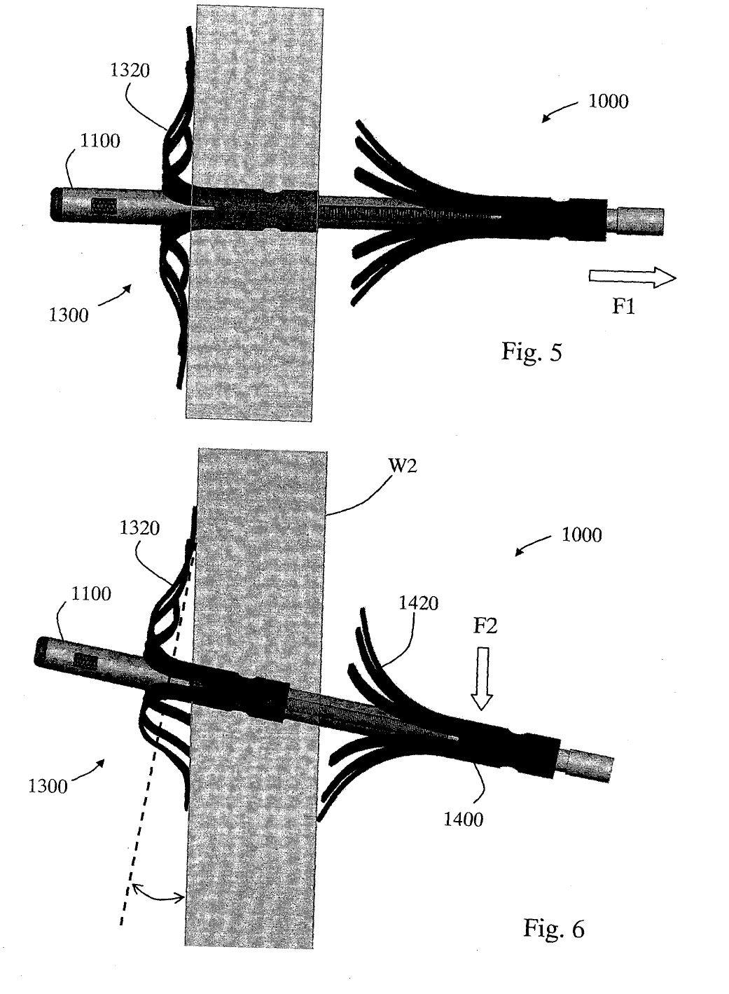

FIG. 5 schematically illustrates implant 1000 having its distal retention member 1300 deformed to a substantially transverse planar shape under a proximally pulling force F1. Pulling forces may be present, for example, if the medical practitioner wishes to check solid retention in-place of implant 1000 or wishes to pull back and withdraw implant 1000 into a delivery device for example in order to relocate it to a different portion in the wall target. In some embodiments, the elastic formation from a first non-stressed shape to the substantially transverse planar shape is applicable under forces F1 only if exceeding 20 grams, optionally exceeding 50 grams, optionally exceeding 80 grams, optionally exceeding 100 grams, optionally exceeding 200 grams, or higher, or lower, or in an intermediate value.

FIG. 6 schematically illustrates implant 1000 implanted through an organ wall shown in a shifted position under a lateral force F2. As shown part of legs 1320 stretch distally and laterally than other legs 1320. Projections 1420 on the other hand are substantially in their non-stressed formation. Lateral force F2 may happen for example if adjacent organs adjacent distal retention member 1400 while wall target shows continuous motility. In such a scenario, when implant 1000 is routinely pushed proximally towards an obstruction, in the form of an adjacent organ or other, it may be forced to tilt away. In some embodiments, implant 1000 and/or retention members 1300 and/or 1400 are sized and configured for maximal tilting of 20% or less, optionally 10% or less, therefore even one leg or a few legs 1320 can support such selective deformation as shown in the figure. In some such embodiments, legs 1320 are at least 4 mm in length, optionally at least 5 mm, optionally about 5.5 mm. Optionally, additionally or alternatively, legs 1320 are shaped and contoured such that free ends 1322 thereof projects horizontally and proximally to point of emergence from base portion 1310 by at least 1 mm, optionally by at least 2 mm, optionally by at least 3 mm, optionally by at least 5 mm, or higher, or lower, or by any intermediate value.

Reference is now made to FIGS. 7A-B which schematically illustrate an exemplary implant 1000′ with retention members shown at different deployment stages via a needle type delivery device 3000. In this exemplary embodiment, implant 1000′ maintains an outer diameter of 1.5 mm or less, for example about 1 mm, and includes self-expandable legs and projections, 4 each. Delivery device 3000 includes a longitudinal tubular member 3100 enclosing a lumen 3200 that is opened at a distal tip 3300. Delivery device 300 may be a catheter based device having member 3100 provided as its distal most portion or member, or alternatively, delivery device 3000 is a needle type device capable of penetrating through tissues, either percutaneously (such as under CT guidance), or directly to the target organ, for example the heart, during open heart surgery. Optionally, distal tip 3300 is sharp and/or beveled to facilitate puncturing capability into soft tissues such as interatrial septum or atrial walls. In some embodiments, lumen 3200 sized for maintaining distal retention member in a fully closed formation when it is provided therein, as shown in FIG. 7A. Implant 1000′ is releasably connectable to a pusher 3500 configured for pushing it outside lumen 3200, thereby releasing the distal retention member from the fully closed formation, and also for pulling it back in lumen 3200, thereby recoverably suppressing the distal retention member to the fully closed formation.

In an aspect of some embodiments, there are provided method for delivering and/or implanting an implant according to the present invention, using distal and proximal retention member. In some embodiments, a method comprises at least one of the followings steps (not necessarily in same order):

(1) locating a wall portion of an heart atrium;

(2) delivering an implant according to the present invention to a target site on an external surface of the wall portion, the implant is provided in a tubular member, the tubular member comprising a lumen that is opened at a distal end thereof and sized for maintaining the distal retention member at a fully closed formation. In some such embodiments, the implant is releasably connected to a pusher;

(3) penetrating with the tubular member through the target site into the heart atrium;

(4) protruding the implant partially outside the lumen using the pusher and/or the tubular member to release the distal retention member from the fully closed formation; and

(5) verifying deployment by applying an axial pulling force to the pusher being greater than substantially 50 grams but smaller than substantially 250 grams, optionally greater than substantially 100 grams but smaller than substantially 200 grams.

In some embodiments, the wall portion is part of a left atrial wall. Optionally and alternatively, the wall portion is part of an interatrial septum and the heart atrium is a left atrium.

In some embodiments, the method comprising also a step of transferring the implant fully outside the lumen using the pusher to release the proximal retention member from a fully collapsed formation.

In some embodiments, the method comprising also a step of retracting the implant from the heart atrium into the lumen by applying a pulling force greater than 100 grams, optionally greater than 250 grams, to the pusher.

In some embodiments, the method comprising also a step of perforating a right atrial wall and passing the tubular member through the perforation into a right atrium.

In some embodiments, the method comprising also at least one of the followings step:
withdrawing the tubular member back through the perforation; and
sealing the perforation.

In some such embodiments, the sealing includes deploying a closure device in or adjacent the perforation. Optionally, the closure device is or includes a second implant according to the present invention.

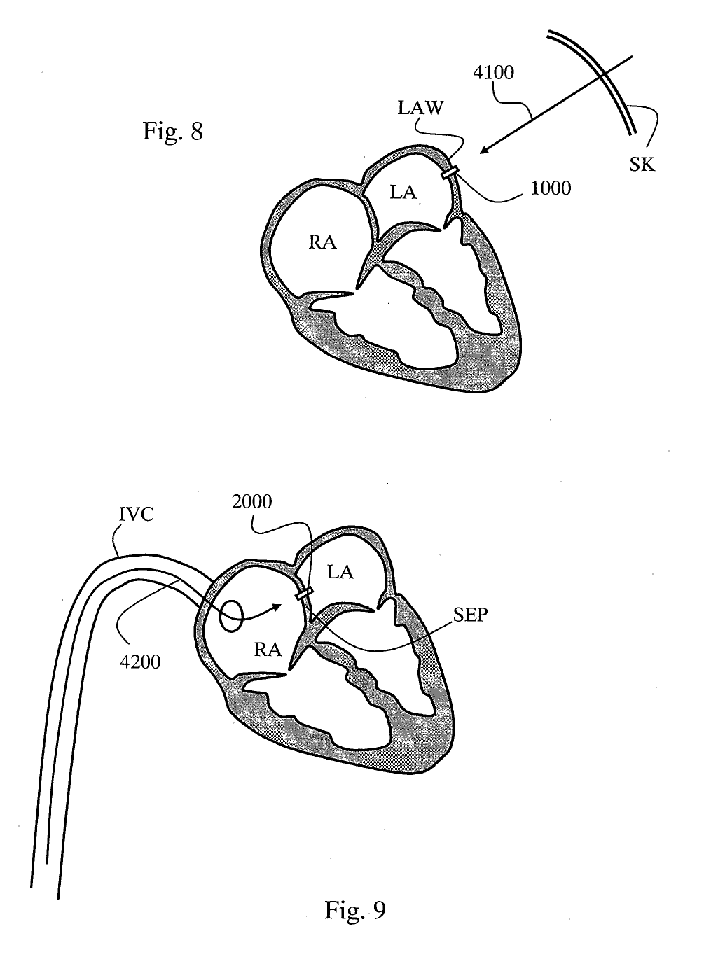

In some embodiments, an implant according to the present invention is configured for measuring pressure in a heart atrium. In some such embodiments, the implant is configured for retention at both ends of a left atrial wall. Reference is now made to FIG. 8 which schematically illustrate an exemplary model for delivering an implant, such as implant 1000, directly to a left atrial wall portion LAW. In some embodiments, implant 1000 and not implant 2000 is preferable as it is advantageous to have “scraper” or “lateral scraping” type proximal retention member, such as 1400, since outer surface of LAW is commonly covered with substantial fat tissue fat which has to be scraped away in order to facilitate correct and axisymmetric expansion of proximal retention member 1400. By using a delivery device such as delivery device 3000 having longitudinal member 3100, preferably provided as a needle type delivery device, the physician may deliver implant 1000 directly to LAW by first opening a chest portion above the heart (not shown) and use delivery device 3000 to puncture through LAW, release distal retention member 1300 by partially pushing pusher 3500 (or pulling back member 3100 while maintaining pusher 3500 in-place), release proximal retention member 1400 by further pushing/pulling until completely removing member 3100 from enclosing proximal retention member 1400. The physician may then disconnect pusher 3500 from implant 1000, for example by unthreading or unbolting. Optionally and alternatively, and as shown in FIG. 8, delivery device 3000 is used for trans-tissue delivery from a percutaneous entry via patient's skin SK along a predetermined straight course 4100 to the outside surface of the target site. Then, actual implantation may proceed as in the open chest surgery. Such methods for delivery and implantation as well as dedicated systems and apparatuses for such delivery and implantation are described in details in PCT/IL2011/050082 to Orion et al., the disclosure of which is incorporated herein by reference in its entirely for all purposes.

In some embodiments, an implant according to the present invention is configured for retention at both ends of an interatrial septum. FIG. 9 schematically illustrates another exemplary model for delivering an implant, such as implant 2000, with a catheter to an interatrial septum SEP portion. Transcatheter approach is a well known technique in which implants can be delivered and anchored to the septum using a dedicated catheter based delivery device maneuverable into the right atrium RA via the inferior vena cava IVC (as in delivering a transcatheter septal occluder for treating atrial septal defects), or optionally the superior vena cava. Optionally and alternatively, the delivery catheter can also be passed into the RA through the aorta (as in delivering prosthetic valves to replace malfunctioned natural valves). Both implants 1000 and 2000 can be used as the SEP is not covered or disturbed by adjacent organs or tissues, as in the case of the LAW, therefore a scraper type proximal retention member is less needed and a “spider's legs” type can be used at both ends of the septum SEP. Once in contact with the SEP, sharp means (such as sharp distal tip 3300 of delivery device 3000) are used to puncture the SEP and penetrate from the RA side to the LA, and implant 2000 can then be deployed as described above. Instead of a dedicated implant such as implants 1000 and 2000, for example a sensory implant, retention devices such as device 2300′, can be used, in which different implants can be fixed either before anchoring or during or after anchoring. In some embodiments, an implant can be deployed using retention members of the present invention having sensors (e.g., pressure sensors) at both ends of implant elongated body so that separate monitoring can be made in RA and LA in-parallel using the same implant or retention device from a single wall target in SEP. Optionally, alternatively or additionally, a separate sensor provided in RA can be used to routinely calibrate pressure reading in LA. Optionally, alternatively or additionally, the RA and/or LA sensors may be calibrated using pressure readings of an external device.

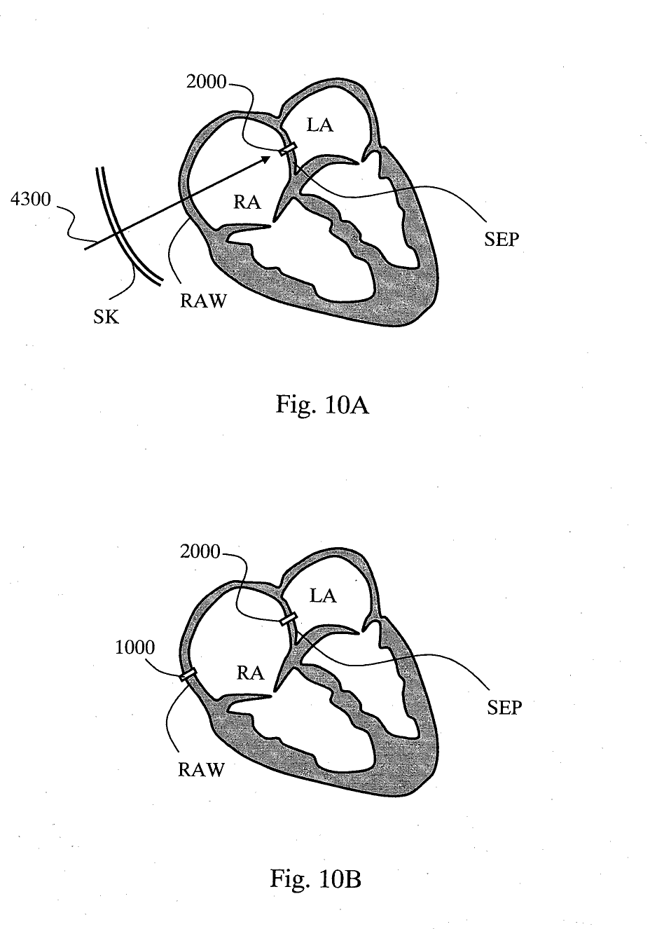

Yet another exemplary model for delivery is shown in FIGS. 10A-B which schematically illustrate exemplary steps for delivering an implant according to the present invention, such as implant 2000, to interatrial septum SEP portion through the right atrial wall RAW. By using a delivery device such as delivery device 3000 having longitudinal member 3100, preferably provided as a needle type delivery device, the physician may deliver implant 2000 to LAW by first opening a chest portion above the heart (not shown), then puncturing, optionally using sharp distal tip 3300, RAW and use delivery device 3000 to further puncture through SEP, release distal retention member 2300 by partially pushing pusher 3500 (or pulling back member 3100 while maintaining pusher 3500 in-place), release proximal retention member 2400 by further pushing/pulling until completely removing member 3100 from enclosing proximal retention member 2400. The physician may then disconnect pusher 3500 from implant 2000, for example by unthreading or unbolting. Optionally and alternatively, and as shown in FIG. 10A, delivery device 3000 is used for trans-tissue delivery from a percutaneous entry via patient's skin SK along a predetermined straight course 4300 to the outside surface of the target site. Then, actual implantation may proceed as in the open chest surgery. Once withdrawing the delivery device from the heart there is a need to seal the puncture in RAW. Different means can be used to seal the RAW puncture, including suturing, gluing (optionally before puncturing) using biological adhesive, coagulating agent and/or hardening material, using a dedicated closure device (optionally deliverable and deployable also with delivery device 3000, optionally in same intrusion), or by deploying another implant or an implant retention device, according to the present invention. As shown in FIG. 10B, implant 1000 may be applied to seal the RAW puncture and optionally may be used to measure pressure in the RA.

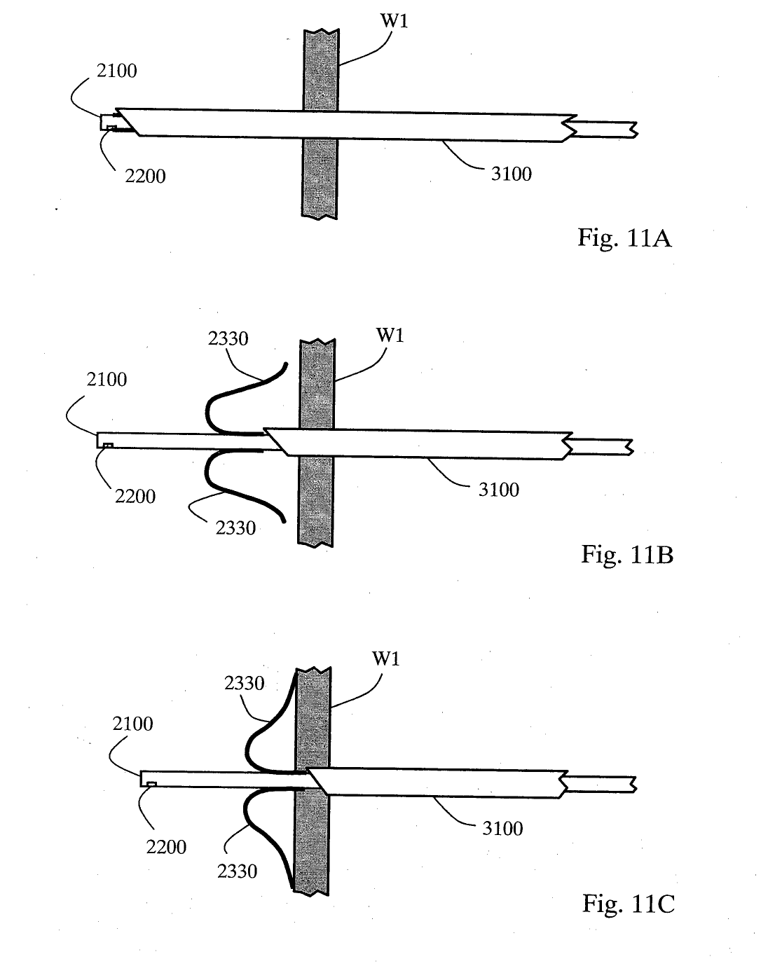

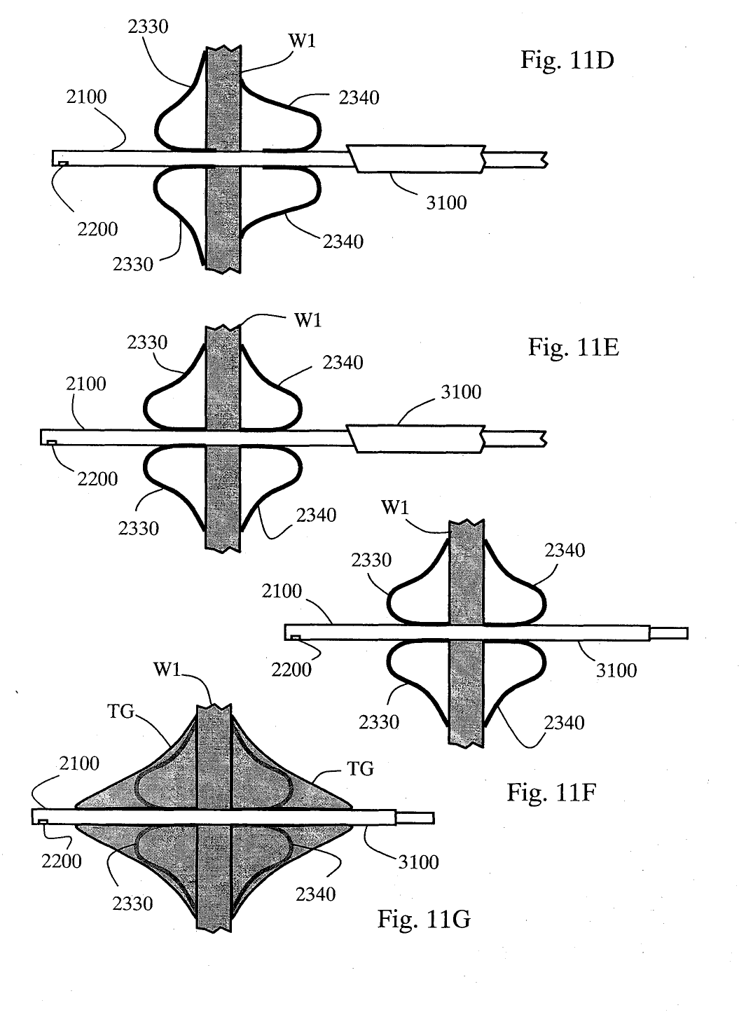

FIGS. 11A-11F schematically illustrate delivery, implantation, and deployment of the cantilevered, symmetrical retention/implant device of FIGS. 2A, 2B and 3 in accordance with an embodiment of the present invention. As described above, the pressure sensing implant 2000 comprises an elongate body 2100 having a proximal end, a distal end, and a lumen therethrough and a pressure sensory element 2200 disposed therein. A flexible proximal retention member coupled to the elongate body 2100 comprising a plurality of projections 2340, each projection having a free end, and a flexible distal retention member coupled to the elongate body 2100 comprising a plurality of legs 2330, each leg having a free end. FIG. 11A illustrates delivery of the implant 2000 via a delivery device, such as sheath 3100, into a heart atrium wall W1 via a transceptal or open heart approach. FIG. 11B illustrates deploying the flexible distal retention member from the delivery device 3100 so that it self-expands from a collapsed configuration, in which the plurality of legs 2330 are substantially straight and extend distally of a base portion 2310, to a non-stressed expanded configuration in which the plurality of legs 2330 extend laterally to the base portion 2310 and proximally. FIG. 11C shows an optional verification step which may be performed by the medical practitioner and includes pulling of elongated body 2100 proximally (backward) in order to check correct anchoring to wall W1 and deployment of legs 2330.

FIGS. 11D and 11E illustrate deploying the flexible proximal retention member from the delivery device so that it self-expands from a collapsed configuration in which the plurality of projections 2340 are substantially straight and extend proximally of the base portion 2310 to an expanded configuration in which the plurality of projections 2340 extend laterally to the base portion 2310 and distally. As shown in FIG. 11F, proximal and distal retention members 2340, 2330 spring towards the base portion 2310 in the expanded configuration so as form symmetrical proximal and distal retention members having spider leg shapes that mirror each other for increased flexibility on both the inner and outer surfaces of wall W1, which is of particular advantage in highly pulsatile/muscular heart wall structures. In particular, the free ends of the proximal and/or distal retention members 2340, 2330 are configured to engage outer and inner surfaces of a left atrial wall or interatrial septum. In some embodiments, the flexible proximal and distal retention members 2340, 2330 are formed from a single retention assembly 2300, wherein the base portion 2310 is coupled to the elongate body 2100. In such a configuration, the plurality of projections 2340 originate at a proximal end of the base portion 2310, each projection 2340 ending with a projection free end and the plurality of legs 2330 originate at a distal end of the base portion 2310, each leg 2330 ending with a leg free end. It will be appreciated that the proximal and distal retention members may also be formed from separate assemblies.

Referring now to FIG. 11G, in some embodiments, the pressure sensing implant 2000 (or any other implant as described herein) is further configured for tissue ingrowth. In particular, the distal retention member is configured for tissue ingrowth TG over said plurality of legs 2330 and/or the proximal retention member is configured for tissue ingrowth TG over the plurality of projections 2340 over time (e.g., 3 months post implantation). In some instances, tissue overgrowth may be achieved free of an ingrowth matrix or the like, wherein the legs 2330 and/or projections 2340 formed from a bare metal, such as super-elastic Ni—Ti alloy, are over grown with tissue over time. This tissue ingrowth TG may further aid in anchoring the sensory implant over long durations of time, which is of particular benefit in highly pulsatile heart wall tissue.

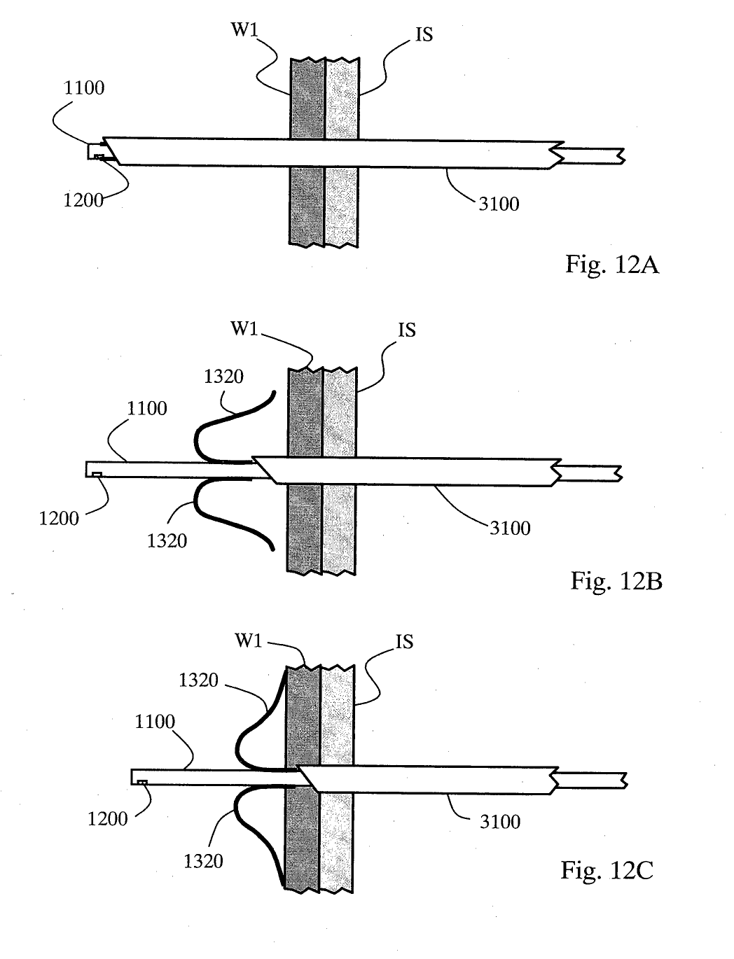

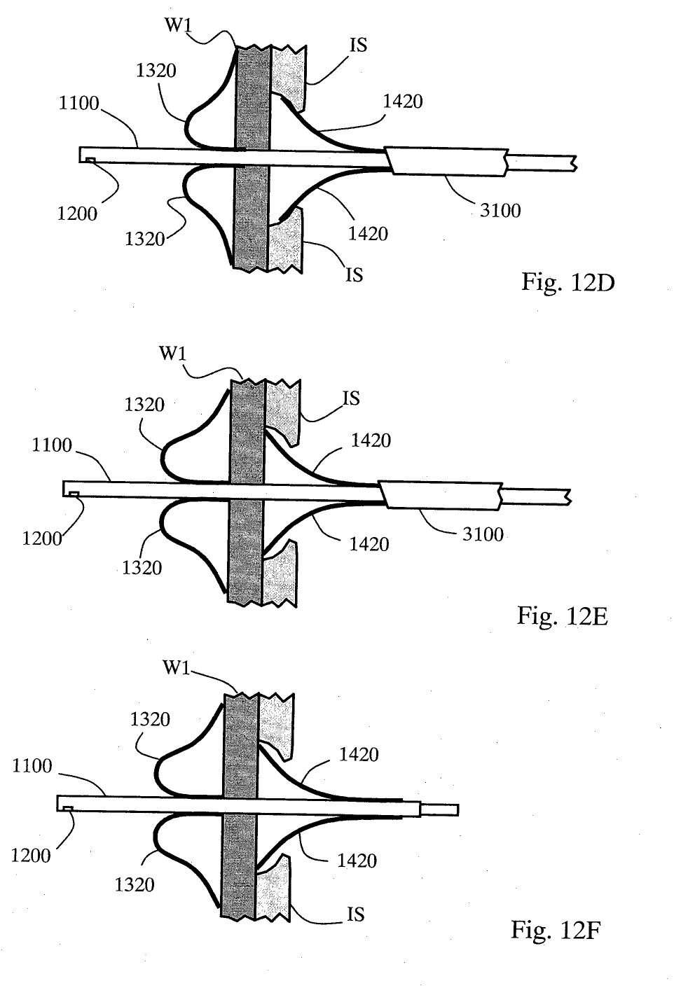

As described above with reference to FIG. 1A, the proximal retention member 1400 may additionally incorporate a scraping function in accordance with another embodiment of the present invention. In some access approaches, it is advantageous to have “scraper” or “lateral scraping” type proximal retention member, such as 1400, since it is configured to move aside, separate, and/or scrape away organs or tissues in order to facilitate correct and axisymmetric expansion of proximal retention member 1400. As shown in FIG. 12A, a needle type of delivery device 3100 penetrates through an intermediate structure IS and target site W1 into a heart atrium. The delivery device 3100 receives and maintains the pressure sensing implant 1100 with proximal and distal legs or retention members in a collapsed configuration. As shown in FIG. 12B, the distal legs 1320 are deployed from the delivery device 3100 so that they self-expand from the collapsed configuration in which the distal legs 1320 are substantially straight and extend axially to a base portion 1310 and distally to an expanded configuration in which the distal retention members 1310 extend laterally to the base portion 1310 and proximally. FIG. 12C illustrates engaging the distal retention members to a first or-inner surface of the target site W1 and verifying proper anchoring and/or deployment of distal legs 1320.

As shown in FIGS. 12D and 12E, the proximal retention members 1420 are deployed from the delivery device 3100 so that they self-expand from the collapsed configuration in which the proximal retention members 1420 are substantially straight and extend axially to the base portion 1310 and distally to the expanded configuration in which the proximal retention members 1420 extend laterally to the base portion 1310 and distally. As shown, the proximal retention members 1420 may be used to scrape or move a portion of the intermediate structure IS away from a second or outer surface of the target site W1. In some embodiments, a free end of each proximal projection 1420 may be configured to scrape or move aside the intermediate structure IS from the target tissue W1. FIG. 12F illustrates engagement of the proximal retention members 1420 to a second or outer surface of the target site W1. The target site W1 may comprise a left atrial wall, wherein the intermediate structure IS may comprise fat or connective tissue, an organ, a left atrial appendage, and like anatomical structures. Advantageously, use of the scraper projection 1420 design allows for improved and forceful opening and anchoring, even under substantial stresses.

FIGS. 13A-13D schematically illustrate deployment and redeployment of a pressure sensing implant with proximal and distal retention members, such as FIG. 1A, 2, or 3. As shown in FIG. 13A, the delivery device 3100, which maintains the pressure sensing implant 1100 with proximal and distal retention members in a collapsed configuration, may penetrate through a first puncture site P1 into a heart atrium via a transceptal or open heart approach. The distal retention member comprising a plurality of legs 1320 originating at a distal end of a base portion 1310 and having free ends may be deployed from the delivery device 3100. The distal retention member may expand from a collapsed configuration in which the plurality of legs 1320 are substantially straight and extend axially to a base portion 1310 and distally to an expanded configuration in which the plurality of legs 1320 extend laterally to the base portion 1310 and proximally. FIG. 13B illustrates re-collapsing the distal retention member by pushing the delivery device 3100 distally over the legs 1320 with a distally oriented force Fd. FIG. 13C illustrates pulling the delivery device 3100 proximally out of the first puncture site P1. FIG. 13D illustrates penetrating the delivery device 3100 through a second puncture site P2 into the heart atrium so that the legs 1320 may be redeployed. It will be appreciated that the proximal projections 1420 may be deployed prior to and/or after the re-collapsing step. For example, the implant design of FIG. 1A advantageously allows for reversibility during or after deployment in case re-positioning is desired. The first or second puncture size P1, P2 may be 2 mm or less in diameter, while the implant size may be 1.5 mm or less in diameter. In some instances, such a small diameter profile allows for the first puncture site P1 to seal naturally. The first or second puncture site P1, P2 may comprise a left atrial wall or interatrial septum wall.

It will be appreciated by those skilled in the art that the system, device and method described above may be used not just for the vascular system and may be applicable to other various organ types, body regions etc.

It will also be appreciated by persons skilled in the art that the present invention is not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

1. A sensory implant comprising:
an elongated body enclosing a lumen and a sensory element disposed therein;
a proximal retention member coupled to the elongated body and comprising a plurality of projections, each of the projections ending with a projection free end; and
a distal retention member positioned distally to the proximal retention member and coupled to the elongated body, the distal retention member comprising a plurality of legs originating at a distal end of a base portion, each of the legs ending with a leg free end,
the proximal retention member and the distal retention member are self-expandable from a fully closed formation, in which the plurality of projections and the plurality of legs extend axially to the base portion to a predetermined non-stressed shape, wherein projection free ends of the projections are configured to move away tissue that interferes with extension or anchoring of the projections.

2-8. (canceled)

9. An implant according to claim 1, wherein each leg at the non-stressed shape includes a first curve, the first curve defines a distally projecting medial angle and a proximally projecting lateral angle.

10. An implant according to claim 9, wherein each leg at the non-stressed shape includes a second curve lateral to the first curve and curved in opposite direction to the first curve.

11. An implant according to claim 10, wherein the first curve includes a first radius of curvature and the second curve includes a second radius of curvature being substantially greater than the first radius of curvature.

12-13. (canceled)

14. An implant according to claim 1, wherein each of the projections at the non-stressed shape includes a third curve, the third curve defines a proximally projecting medial angle and a distally projecting lateral angle.

15. An implant according to claim 14, wherein each projection at the non-stressed shape includes a fourth curve lateral to the third curve and curved in opposite direction to the third curve.

16. An implant according to claim 15, wherein the third curve includes a third radius of curvature and the fourth curve includes a fourth radius of curvature being substantially greater than the third radius of curvature.

17. (canceled)

18. An implant according to claim 1, wherein each of the projections at the non-stressed shape is substantially straight or is curved in a single direction only.

19. An implant according to claim 1, wherein any of the distal retention member and the proximal retention member is formed as a single structural part.

20. An implant according to claim 19, wherein the single structural part is tubular and wherein the forming includes creating longitudinal slits, thereby each two adjacent slits define a leg.

21. An implant according to claim 20, wherein the single structural part is a Ni—Ti alloy in a super-elastic condition.

22. (canceled)

23. An implant according to claim 1, wherein the legs are elastically formable from the first non-stressed shape to a substantially transverse planar shape.

24-29. (canceled)

30. An implant according to claim 1, wherein the sensory element extends along a side and in proximity to a distal end of the elongated body.

31-32. (canceled)

33. An implant according to claim 1, configured for retention at both ends of a left atrial wall.

34. An implant according to claim 1, configured for retention at both ends of an interatrial septum.

35. (canceled)

36. An implant according to claim 1, provided for delivery to a target site in a tubular member comprising a lumen opened at distal end thereof and sized for maintaining the distal retention member at the fully closed formation.

37-53. (canceled)

54. An implant according to claim 1, wherein the projection free ends in the non-stressed shape have straight edges, which point toward the tissue and are configured to recollapse to the fully closed formation for removal of the implant.
